(19) United States
(12) Patent Application Publication
(10) Pub. No.: US 2004/0244807 A1
(43) Pub. Date: Dec. 9, 2004

(54) Sleep-lab systems and methods

(76) Inventors: Jianguo Sun, San Mateo, CA (US); Laughton Miles, Los Altos Hills, CA (US); Victor Prupis, Palo Alto, CA (US); Julia Tverskaya, Palo Alto, CA (US); Kang Zau, San Jose, CA (US)

Correspondence Address: LATHROP & GAGE LC, 4845 PEARL EAST CIRCLE, SUITE 300, BOULDER, CO, 80301, US

(21) Appl. No.: 10/861,089

(22) Filed: Jun. 4, 2004

Related U.S. Application Data
(60) Provisional application No. 60/476,213, filed on Jun. 4, 2003.

(51) Int. Cl. A61B 5/00

(52) U.S. Cl. 128/904; 600/300; 709/213; 709/219

(57) ABSTRACT

Systems and methods are provided for operation of a sleep-lab. A central processor provides connectivity between sleep-lab personnel, sleep-lab equipment, monitoring and analysis personnel and the patient. The sleep-lab supports at-home treatment and diagnostics, monitor-only sleep labs, and conventional sleep-labs. Monitoring and analysis, performed by the real-time monitor, scorer and over-reader, interpreter, and follow-up analyst, may be performed at a central location or disbursed throughout the world to take advantage of time-shifting and/or other resources. A central processor provides security and connectivity to facilitate the data exchange between data sources and monitoring and analysis facilities. A sleep-lab communication protocol helps to ensure a loss-less transfer of data over an interruptible network connection. A sleep-lab interface provides data conversion and accounting processes.

### CROSS REFERENCE TO RELATED APPLICATIONS

This application claims priority to U.S. provisional application Ser. No. 60/476,213, filed 4 Jun. 2003 and hereby incorporated by reference.

### BACKGROUND

In typical sleep-lab operations, a patient is referred to a sleep-lab for evaluation by the patient's physician. The patient contacts a sleep-lab and schedules an overnight sleep-study. The scheduled night must correspond to the availability of the sleep-lab's personnel, facilities and equipment, as well as the patient's ability to be away overnight. It is common for patients to wait over four weeks for an available time slot in many sleep-labs.

Upon arrival to the sleep-lab, the patient is familiarized with sleep-lab testing, equipment and environment. Before testing begins, the patient is connected to monitoring sensors and taken to a sleep room. After the sensors and/or devices are connected and verified, the patient is left to sleep. During a split-night study, a treatment device (e.g., a Continuous Positive Airway Pressure device; “CPAP”) is connected to a patient during the second half of the study to determine therapeutic pressure. Trying to sleep while attached to monitoring and treatment equipment is difficult, particularly for first time patients. Compounding the problem is the unfamiliar bed and surroundings of the sleep-lab. As a result, the sleep-study may not have the opportunity to record and evaluate a typical night's sleep.

The sleep-study is typically costly. A sleep-lab must be able to accommodate sleep rooms, monitoring equipment and staff. For example, a sleep-lab typically requires a single overnight technician for every 2 patients. The location of the sleep-lab must be reasonably convenient for the patients and staff and must also minimize a patient's sleep interruptions. The location also determines, in part, the cost required to acoustically modify the sleep-lab. For example, in metropolitan areas, or other areas where real estate values are costly, sleep-labs face competition for facilities. As a result, the facility cost of a sleep-lab may be quite high, despite the limited use of the facility during regular business hours.

Typically sleep-labs require skilled, on-site personnel to monitor patients overnight. The cost of these skilled, overnight technicians further increases the cost of sleep-studies. The availability of such technicians can limit or curtail operations, to meet budget constraints. As a result, sleep-labs often attempt to pass costs to the patient's insurance company, pass unreimbursed costs to the patients and/or subsidize sleep-lab operations with other services.

Many patients do not have ready access to a sleep-lab and must therefore travel to find a suitable facility. Such a travel burden often limits a person from accessing a sleep lab. The burden is exacerbated if multiple sessions are required; as is typically the case to facilitate follow-up care or post-treatment evaluation.

Sleep-studies are generally performed at sleep-labs (e.g., free standing, hospital outpatient, or at-home facilities). Sleep-labs provide monitoring, data processing, analysis, treatment, follow-up analysis and treatment adjustment within the same facility. At-home facilities are generally limited to screening tests, specific treatments, and limited or no monitoring.

These typical sleep-lab operations impose many burdens on the providers and patients. Ultimately, patients may endure travel burdens based on sleep-lab locations and scheduling restrictions of the sleep-lab. Additionally, a portion of the medical staff must work overnight hours which increases costs. Sleep-lab owners must balance the facility costs and the location costs with patient and staff accessibility. As a result of these and other burdens, common sleep-lab operations are resource inefficient.

### SUMMARY

The present disclosure solves certain of the above and other problems and advances the state of the useful arts by providing sleep-lab methods and systems. By way of example, the burdens imposed on sleep-lab patients may be reduced and provide the patients with improved access to sleep disorder diagnosis and treatment. As a result of improved accessibility, the diagnosis and treatment of sleep disorders can be expanded to more individuals to promote a healthier population.

By way of example, methods for operating a virtual sleep-lab are provided for sleep-lab monitoring, analyzing, diagnosing, treating, and re-evaluating a patient. Such sleep-labs may be self-run, satellite and/or at-home facilities. In another example, methods and systems are provided for a sleep-lab to monitor, score and over-read, interpret and follow-up (e.g., CPAP follow-up) a patient in a virtual sleep-lab. Scoring is performed by a polysomnographic technologist scoring the sleep-study as one component of sleep-study analysis. The analysis may also include defining sleep architectures, calculating respiratory events and/or other analysis functions. Over-reading is a function performed by a sleep specialist reviewing the sleep-data and scoring results. Interpretation is a diagnostic report created by the sleep specialist. Interpretation is based on patient information and the outcome of the sleep-study.

In one embodiment, a sleep-data transmission system is provided. The sleep-data may be sent between a host and a client over a network. The host contains a data fragment database containing each segment of the sleep-data. The sleep-data includes one or more data fragments. In one example, a datamap is an index of the database and readily indicates present and missing data fragments. The host may request a missing data fragment from the client, receive the requested data fragment and update the datamap and database accordingly. The client may also contain a data fragment database. In one example of operation, the client data fragment database is indexed by a client datamap indexing a client database of data both successfully and unsuccessfully transferred to the host. In an example of operation, the host identifies a missing data fragment and requests the missing data fragment from the client. The client may then send the data fragment such that the host updates the host datamap and database to indicate the presence of the data fragment. The host may then confirm receipt to the client such that the client may update the client datamap and database to reflect successful transfer of the data fragment to the host. As a design option, the client datamap may be updated to reflect transfer to a plurality of hosts or to omit updating.

In one embodiment, a data transmission protocol is provided for the transfer of sleep-data. In one example of operation, the host requests a missing data fragment description from the host datamap. The host datamap replies with either a description of the missing data fragment (e.g., a file name, a date, a client name, a length and/or a format) or an indication that there are no missing data fragments. If the host receives a description of a missing data fragment, the host may then request the missing data from the appropriate client. The client may forward the request for the missing data fragment to the client datamap. The client datamap may return either a detailed description of the data fragment (e.g., a file name, a position in the client datamap, a position in the database, an actual record time, an actual file length, an actual file size) with the data fragment or an indicator of unavailability of the data fragment. In one example, the unavailability of a data fragment further indicates the data fragment's unavailability is permanent or temporary. The client returns the result received from the client datamap to the host. The host is then able to update the host datamap and database with either the data fragment received or a status of unavailability. In another example of operation, the process is idle for a predetermined length of time and repeats until all available data fragments (e.g., data fragments that are not permanently unavailable) have been received.

In one embodiment, a system for remote sleep-lab patient monitoring is provided. In one example, a patient is connected to a biometric sleep monitor to read patient physiology, such as heart rate, blood pressure, blood oxygen level, respiratory rate, brain activity, muscle activity, limb movement and/or sleep position. The sleep monitor records raw sleep-data for transmission to monitoring and processing equipment and personnel over the network. A central processor receives raw sleep-data and limits access to raw and processed sleep-data to authorized systems and personnel. The central processor directs the flow of sleep-data and ancillary data (e.g., commands to adjust a device, alert processing data and/or medical staff communications). The raw sleep-data is received by the central processor which then grants access to the raw sleep-data for authorized monitors and processors (e.g., scorers, interpreters, over-readers and/or analyzers). The processors may be systems and personnel centrally located or widely distributed within a sleep-lab operation. In one example of operation, a patient is monitored overnight in one location while real-time monitoring occurs in a second location, which may be time-shifted (e.g., in a different time zone) to perform overnight patient monitoring during substantially non-overnight hours. Scoring may be performed at another location; over-reading may be performed at yet another location; and analysis may be performed at still another location. The central processor is operable to establish and to manage communication channels, to receive and to store raw data and processed data, to authorize access to data, to limit or to grant access to devices and/or to facilitate communication.

In one embodiment, a real-time monitor monitors a sleep-study patient by receiving data, over a network, from sleep-study monitoring equipment that has sensors configured for receiving biometric information from the patient. Sleep-study monitoring equipment provides detailed monitoring of a patient biometrics in substantially real-time. Raw sleep-data produced by sleep-study monitoring equipment is often voluminous and transmission of the sleep-data may overwhelm a network having limited bandwidth. Data may be received in true real-time, in near real-time or in batch mode to accommodate limitations and interruptions of the network and other business objectives. The real-time monitor processes data as it is received.

The monitoring equipment may upload raw sleep-study data to a central processor. As an option, data transfer between the monitoring equipment and a central processor is facilitated by a virtual sleep-lab (“VSL”) remote sleep monitoring system, such as the VSL Box or VSL Hub described below. In one embodiment, the network is a proprietary network, or is one of a combination of known networks (e.g., direct connection, dial-up, infrared, wireless and/or Internet) that provides data connectivity between the monitoring equipment and the central processor.

In another embodiment, a VSL Box connects a patient's on-site sleep-study monitoring equipment to the central processor. The VSL Box is, for example, a personal computer with specialized software or a dedicated hardware device, such as an embedded system or integrated circuit. The VSL Box may provide a secure data portal between an authorized user on the network, such as the patient's physician, the real-time monitor and/or sleep-study monitoring equipment. As an optional embodiment, the VSL Box may provide temporary data storage of data received from the sleep-study monitoring equipment until the data is transferred to the central processor. The VSL Box may also manage network connectivity and establish, or re-establish, data transfer sessions with the central processor. As a further option, the VSL Box may provide audio, video, text and/or iconic communication (e.g., illumination of an LED, highlight a button on a touch-pad, etc.) with the patient or the patient's on-site monitor (e.g., an overnight technician and/or a family member). In one embodiment, the VSL Box receives commands from an authorized user to be executed by the sleep-study monitoring equipment. Sleep-study equipment may be operated or modified by off-site personnel.

In one embodiment, a VSL Hub connects a patient's home treatment and monitoring equipment and the central processor. Home treatment and monitoring equipment (“follow-up”) includes devices, such as heart monitors, respiratory monitors, blood-oxygen sensors and CPAP devices. Follow-up equipment may produce less data and therefore may require fewer network resources compared to sleep-study monitoring equipment. The VSL Hub provides a more economical utilization of network resources (e.g., store-and-forward mode) than the VSL Box, which typically uses real-time data transmission. As a design option, the VSL Hub and/or VSL Box may receive and store “quality of life” information, such as how rested a patient feels and/or perceptions of sleep improvements. The VSL Hub may provide some or all of the VSL Box functionality.

In one embodiment, a method of real-time monitoring is provided. Real-time monitoring may be performed on-site (e.g., substantially co-located with the patient) or off-site. Sleep-study or follow-up data is conveyed from on-site equipment to the central processor and from the central processor to the real-time monitor. The real-time monitor, for example, is a technician watching and listening for cues on a workstation with specialized software. The specialized software presents sleep-study and/or follow-up data to the technician in a form that alerts the technician of events. As an option, the technician is an expert system or other artificially intelligent system that may supplement or replace a human technician. Real-time monitoring may trigger an event response from the technician. The responses include annotating the data, alerting on-site personnel, alerting the patient, alerting another real-time monitor, alerting other off-site personnel, summoning an emergency response team, issuing a command to a VSL Hub or VSL Box and/or issuing a command to the on-site equipment. The central processor is operable to provide or eliminate event response options available to the technician. For example, the central processor eliminates event response options not approved by the patient's physician, not within the operating parameters of the on-site equipment and/or not allowed by accepted medical practice. If approved, the network conveys the command or alert to the appropriate recipient. As a design option, sleep-studies may be monitored by a plurality of real-time monitors simultaneously.

Certain benefits may be obtained with use of the foregoing systems and methods. In one example, a technician is located in a time-zone that facilitates monitoring of overnight patient studies such that the technician works substantially no overnight hours. In another example, a patient is evaluated by one or more skilled medical professionals without regard to the location of the professionals, either collectively or individually. Such professionals include remote monitors, scorers, over-readers, sleep-data interpreters, follow-up analysts and/or other interpretive or diagnostic personnel.

In one embodiment, a scorer and an over-reader provide scoring and over-reading, respectively, of sleep-study and follow-up data. A central processor grants authorized scorers and over-readers access to patient data.

In another embodiment, a sleep-data interpreter interprets raw sleep-data and/or previously analyzed (e.g., scored) sleep-data. A central processor grants authorized sleep-data interpreters access to patient data.

In one embodiment, a follow-up analyst evaluates patient data to recommend refinements to patient treatment. A central processor grants authorized follow-up analysts access to patient data.

In one embodiment, a central processor allows for real-time and non real-time communication of messages and events among one or more of a patient, a real-time monitor, a scorer, an over-reader, a sleep-data interpreter, a follow-up analyst and/or other authorized systems or personnel. Secure collaboration and event notification is provided between co-located and non co-located individuals and systems.

In one embodiment, a central processor provides communication and loss-less data transmission of data. The central processor is one or more physical servers, on dedicated or shared equipment, in one or more physical locations to provide a logically centralized processor. The central processor executes software applications to support network connectivity, data transmission, security, data presentation, sleep-study or follow-up equipment identification and operating parameters, patient data storage and retrieval, device command rules, alert processing rules and/or personnel authorizations. The data transmission and presentation software may include a session manager, a download server (“downloader”), an upload server (“uploader”), a fragment database and/or a web server and has a host component operable on the central processor and a client component operable on a client device. The client device may include sleep-study and/or follow-up equipment, such as a VSL Box or a VSL Hub. In one example of operation, the session manager controls and manages user access, network connectivity and persistent sessions across any disrupted connections.

In one embodiment, an upload server responds to requests for data from a client device. In one example, the upload server initiates a new session for each attached client device. In another embodiment, a download server collects recordings from either sleep-study equipment or a VSL Box attached to the sleep-study equipment. An upload server controls network access to sleep-study data on the sleep-study equipment or the VSL Box. The upload server may maintain a client datamap of data fragments wherein each data fragment contains a portion of sleep-data. Optionally, the client datamap contains placeholders for data fragments, either successfully uploaded or unavailable. The downloader may maintain a host datamap of data fragments of successfully uploaded data fragments. The host datamap may optionally contain placeholders for data fragments, either permanently or temporarily unavailable. The download server attempts to collect the “current” data fragment from each client. However, if network bandwidth and other resources allow, a background process can be initiated to download previously missed data fragments. After recording is complete the download server may request any data fragments not previously retrieved. If no data fragments are retrievable, the transfer session terminates.

### BRIEF DESCRIPTION OF DRAWINGS

FIG. 1 shows one sleep-lab system;

FIG. 2 shows one data transfer process to request missing sleep-data;

FIG. 3 shows one data transfer process to request a sleep-data fragment;

FIG. 4 shows one data transfer process to open a sleep-data fragment;

FIG. 5 shows one data transfer process to close a sleep-data fragment;

FIG. 6 shows one data transfer process to close a sleep-data fragment datamap; and

FIG. 7 shows one data transfer process to retrieve a sleep-data fragment.

### DETAILED DESCRIPTION OF DRAWINGS

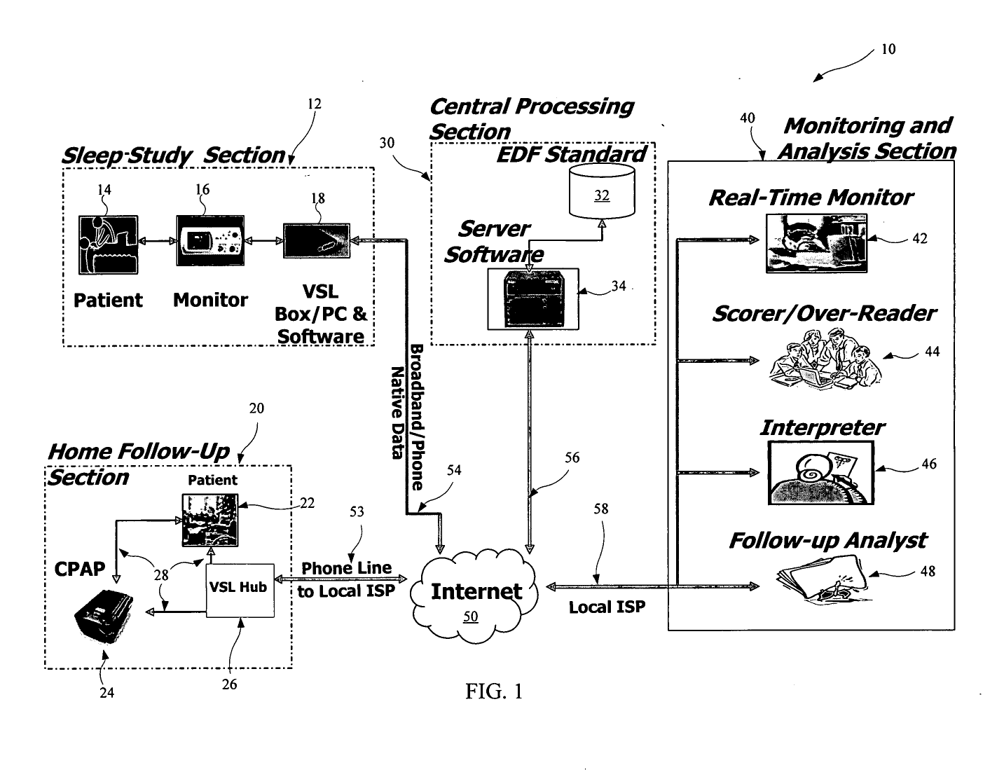

FIG. 1 shows one sleep-lab system 10. Sleep-lab system 10 includes a sleep-study section 12, a home follow-up section 20, a central processing section 30, a monitoring and analysis section 40, and a network 50, which in this embodiment is exemplary of the internet. A physical location for sleep-lab system 10 may be the location of sleep-study section 12, home follow-up section 20, central processing section 30, monitoring and analysis section 40, or network 50. Optionally, sleep-lab system 10 may be situated at a location with real-time monitoring 42 as a matter of design choice to accommodate facility resources, and/or other medical/business objectives.

Sleep-study section 12 has monitoring equipment 16 used to monitor patient 14. Monitoring equipment 16 may connect directly to network 50, through connection 54, and provide secure network connectivity. Alternatively, a VSL Box 18 may provide temporary data storage and/or secure network connectivity to network 50. Monitoring equipment 16 may be unidirectional (e.g., to record sleep-study data) or bidirectional (e.g., to record sleep-study data and to issue commands to monitoring equipment 16) in its communication with VSL Box 18. Connection 54 may be a dedicated connection, dial-up, Internet and/or other network bus or medium as a matter of design choice. VSL Box 18 also receives commands from authorized users through connection 54. In turn, VSL Box 18 either executes the received command or forwards the command to monitoring equipment 16. In one example of operation, the in-home sleep-labs wake patient 14 to request that patient 14 perform an action, such as reattaching a dislocated sensor providing input to monitoring equipment 16. In another illustrative example, an on-site sleep-lab, such as a full-service or a satellite lab, may alert an on-site monitor, such as real-time monitor 42, to perform a requested operation.

Home follow-up section 20 provides at-home monitoring. A VSL Hub 26 may enable communication between at-home follow-up equipment, such as a CPAP device 24, and monitoring and analysis section 40. Additionally, VSL Hub 26 may provide communication connectivity between patient 22, monitoring and analysis section 40 and/or CPAP device 24. In one example, VSL Hub 26 stores data and commands for later communication with monitoring and analysis section 40. As an option, a patient 22 interacts directly with CPAP device 24 or uses VSL Hub 26 to interface with CPAP device 24 or other devices. In one example, a simplified user interface of VSL Hub 26 provides a single point of control for single or multiple at-home devices. VSL Hub 26 can provide patient 22 with alerts or informative messages sent from CPAP device 24 or monitoring and analysis section 40. Similarly, patient 22 and/or CPAP device 24 may utilize VSL Hub 26 to send and receive alerts or informative messages to monitoring and analysis section 40.

Central processing section 30 contains a server 34 to provide authorized human and electronic users of system 10 with real-time and stored data. As an option, communication within system 10 is restricted to authorized users' determined by central processing section 30. As a further option, secure communications, such as when real-time monitor 42 and VSL Box 18 are co-located, are permitted without approval of central processing section 30. As a design option, data is stored external to server 34 in a central repository 32. Central repository 32 may provide multiple data formats and conversion routines. One example of a data format may include the European Data Format (“EDF”) standard. Server 34 facilitates communication by providing required routing data and rules for communication. An example of routing data is an Internet Protocol (“IP”) address of a device, such as VSL Box 18. An example of a rule for communication is use of a stored schedule of on-call physicians to determine which on-call physician's pager number should be dialed. Another example of a rule for communication determines how to execute a command; for instance, if one or more sensors of sleep-lab monitor 16 have become detached from patient 14, server 34 may determine that an alert is required to request reattachment. If patient 14 is at home, server 34 may issue the alert to VSL Box 18. However, if patient 14 is at an on-site sleep lab, a human monitor, such as real-time monitor 42, may be alerted by sending a message to a workstation or pager.

Monitoring and analysis section 40 provides real-time and/or historical monitoring of patient data. Monitoring and analysis section 40 can be further subdivided into a real-time monitor 42, a scorer and over-reader 44, an interpreter 46, and a follow-up analyst 48. Interpreter 46 is a technician or electronic system providing analysis of patient data. In one example, follow-up analyst 48 is a technician specializing in the analysis and adjustment of patient treatment and treatment devices, such as CPAP 24. Real-time monitor 42, scorer and over-reader 44, interpreter 46, follow-up analyst 48, central processing section 30 and patient 14 or 22 may be located independently of each other. As an option, any of real-time monitor 42, scorer and over-reader 44, interpreter 46, and follow-up analyst 48 may be one or more human users, computer programs, artificially intelligent systems and/or combinations thereof.

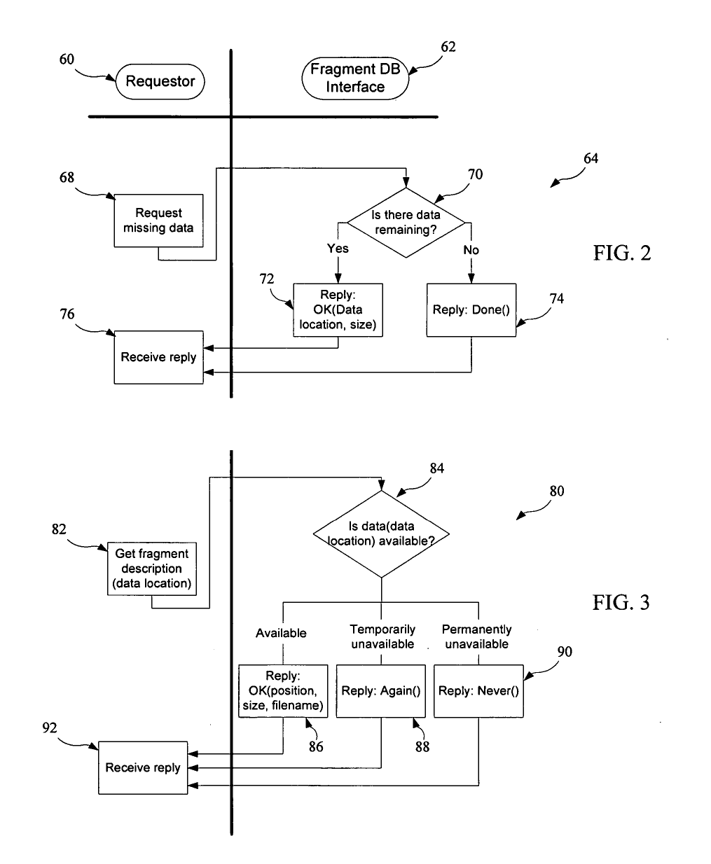

FIG. 2 shows one exemplary data transfer protocol (64) to request missing data for use with the system of FIG. 1. A requestor 64, which may be functionally operational on central server 34, requests (68) any missing data fragments from a fragment database 62. A test 70 determines if there is a missing data fragment. If there is, a “yes” reply is created (72) with a data location and a size of the missing data fragment; if there is not, a “no” reply is created (74) to indicate no data has been identified as missing. Requestor 60 then receives (76) the corresponding reply from fragment database 62.

FIG. 3 shows one exemplary data transfer protocol (80) to request a data fragment description for use with the system of FIG. 1. A requestor 60 creates (82) a request containing a data fragment location and forwards the request to fragment database 62. A test (84) determines whether the data exists at the specified location, and if so creates (86) an “OK” reply with a position, a size and a file name of the data fragment. If test (84) determines the data, at the specified location, is temporarily unavailable, then an “Again” reply is created (88). The “Again” reply (88) indicates that no data is being returned and that the host should re-request the data fragment. If test (84) determines the data at the specified location is permanently unavailable, then a “never” reply is created (90). The “never” reply (90) indicates that no data is being returned and the host should not re-request the data fragment. Requestor 60 receives (92) the reply from fragment database 62.

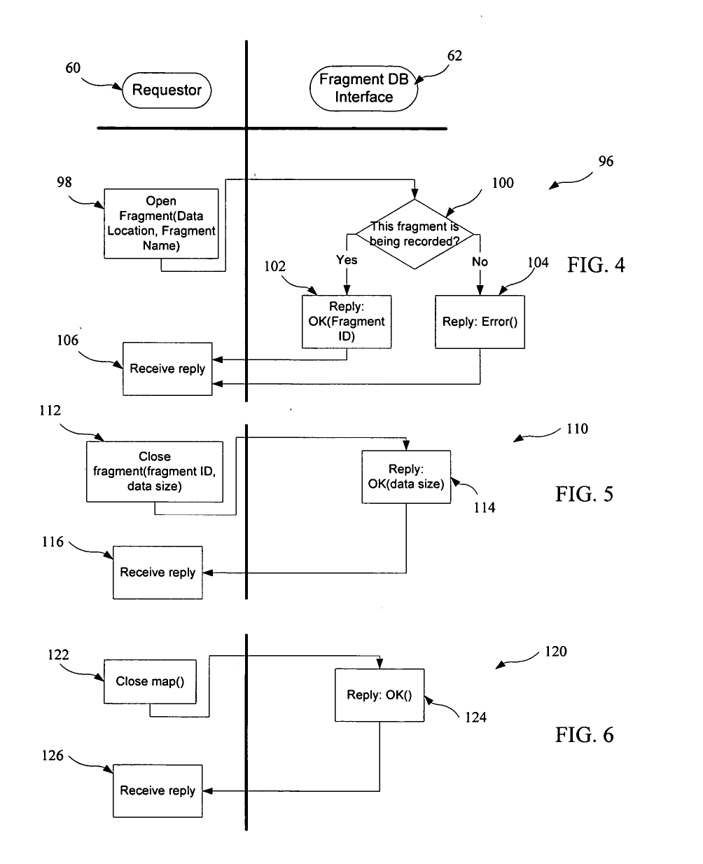

FIG. 4 shows one exemplary data transfer protocol (96) to open a data fragment for use with the system of FIG. 1. Data transfer protocol (96) may be utilized to query data fragment database 62 to determine the validity of a specified data fragment. Requestor 60 formats (98) a request with a data location and a fragment name for fragment database 62. If test 100 determines that the data fragment is currently being recorded, then a reply may be formatted (102). The reply created (102) returns “OK” with a fragment ID. If test 100 determines that the data fragment is not currently being recorded, then an error reply may be formatted (104). The error reply (104) may return an error value to indicate the data fragment has already been transferred to requestor 60. Requestor 60 receives reply (106) from fragment database 62.

FIG. 5 shows one exemplary data transfer protocol 110 to close a data fragment for use with the system of FIG. 1. Requestor 60 creates (112) a request with a data fragment identification and a first data size and sends the request to fragment database 62. The first data size may provide a desired, post-truncated, file size of the data fragment. The first data size may be less than an actual data fragment size when, for example, a previous attempt to load the data fragment was partially successful. The post-truncated file size may correspond to a specific size of the data fragment in requestor's 60 datamap. A reply is created (114) with a second data size. The second data size may be a valid data fragment size provided after an attempt to truncate the data fragment to the first data size. Requestor 60 receives (116) the reply from fragment database 62.

FIG. 6 shows one exemplary data transfer protocol (120) to close a data fragment map for use with the system of FIG. 1. Requestor 60 creates (112) a request and sends the request to fragment database 62. Fragment database 62 creates (124) an “OK” reply. Requestor 60 receives 126 the reply from fragment database 62.

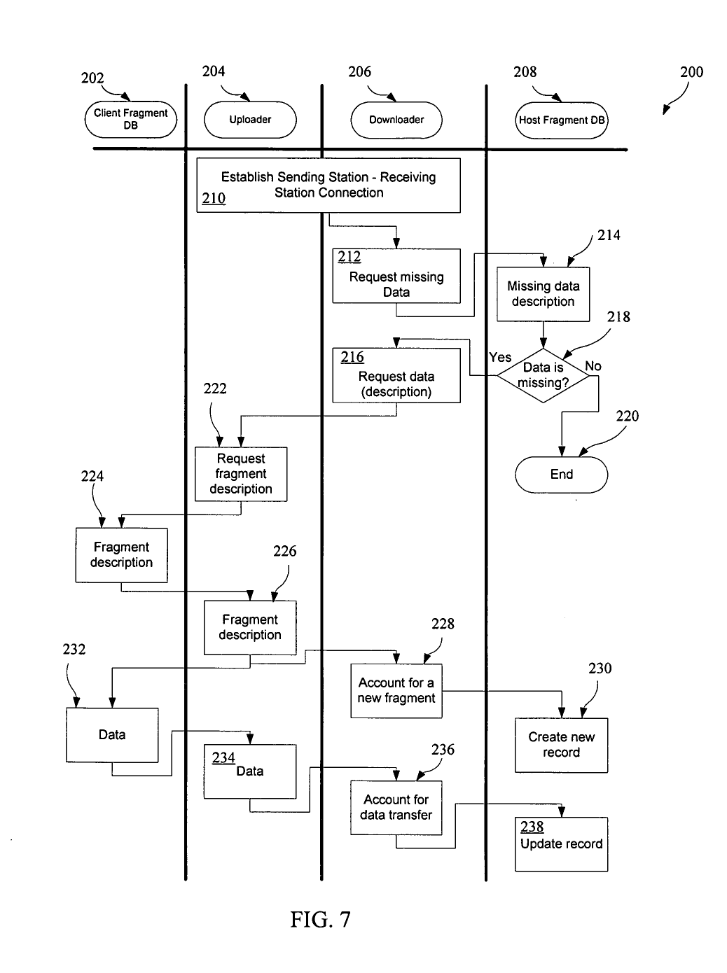

FIG. 7 shows one exemplary data transfer protocol (200) to retrieve data from a client device, such as the VSL Box, VSL Hub, monitoring equipment and/or treatment equipment for use with the system of FIG. 1. In one example, a client fragment database 202 and an uploader 204 are co-located on the same client device. A downloader 206 and a host fragment database 208 may be co-located on a central server, such as server 34. A data connection is established (210) between the client device and the central server. Downloader 206 requests (212) missing data from host fragment database 208. The missing data description is received (214) and tested (218) to determine if a data fragment is missing. If test (218) determines that the data fragment is not missing, protocol 200 terminates (220). If test (218) determines the data fragment is missing, a data request is formatted (216) and sent to uploader 204. Uploader 204 forwards (222) the data request to client fragment database 202. Client fragment database 202 formats (224) a data fragment description for uploader 204. Uploader 204 formats (226) a data fragment description and sends the description to downloader 206. Downloader 206 accounts (228) for the new data fragment, which may include updating a datamap. Host fragment database 208 then creates (230) a new record for the data fragment. The actual transfer of data may be initiated by a separate process or integrated into the formatting (226) as a matter of design choice. The data fragment is then transferred (232) from client fragment database 202 to uploader 204. The data fragment is then transferred (234) to downloader 206, which accounts (236) for the actual data transferred. Host fragment database 208 then updates (238) the datamap record created (230). To further illustrate protocol 200, data is generally transferred from a client device to a host in manageable increments (i.e., data fragments) with near certainty that a complete data record will be transferred completely, regardless of the number or frequency of network interruptions.

Changes may be made in the above methods and systems without departing from the scope hereof. It should thus be noted that that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

What is claimed is:

1. A system for remote sleep-lab patient monitoring comprising:
a patient connected to a biometric sleep monitor located at a first location wherein the biometric sleep monitor reads the patient physiology as raw sleep-data;
the biometric sleep monitor is connected to a network, providing raw sleep-data to a central server at a second location;
an analyzer at one or more other locations connected to the network, receiving raw sleep-data from the central server and providing analyzed data.

2. A system of claim 1, further comprising biometric sleep monitor receive data and commands from an authorized user of the network connection.

3. A system of claim 1, wherein the biometric sleep monitor is connected to the network through a connection with a virtual sleep-lab connectivity device.

4. A system of claim 1, wherein the biometric sleep monitor provides a treatment to the patient.

5. A system of claim 1, wherein the analyzer at one or more other locations further comprises:
a first monitor of the raw sleep-data at a third location connected to the network to receive raw sleep-data, monitor the raw sleep-data and issue commands.

6. A system of claim 5, wherein the first monitor issues commands to be received by one of the biometric sleep monitor and the virtual sleep-lab connectivity device.

7. A system of claim 5, wherein the first monitor issues commands to be received by a device capable of conveying the command to a healthcare provider at the first location.

8. A system of claim 5, wherein the first monitor issues commands to be received by the central server.

9. A system of claim 5, wherein the first monitor issues commands to be received by another authorized recipient of the commands.

10. A system of claim 5, wherein the analyzer at one or more other locations further comprises:
a second monitor of the raw sleep-data at a fourth location connected to the network to receive raw sleep-data, monitor the raw sleep-data, and issue commands.

11. A system of claim 10, wherein the second monitor issues commands to be received by one of the biometric sleep monitor and the virtual sleep-lab connectivity device.

12. A system of claim 10, wherein the second monitor issues commands to be received by a device capable of conveying the command to a healthcare provider at the first location.

13. A system of claim 10, wherein the second monitor issues commands to be received by one of the first monitor and the central server.

14. A system of claim 10, wherein the second monitor issues commands to be received by another authorized recipient of the commands.

15. A system of claim 10, wherein the first monitor issues commands to be received by the second monitor.

16. A system of claim 1, wherein the analyzer at one or more other locations further comprises:
a scorer of the raw sleep-data at a third location connected to the network to receive raw sleep-data, score the raw sleep-data, and issue a scored sleep report to authorized recipients on the network.

17. A system of claim 1, wherein the analyzer at one or more other locations further comprises:
an interpreter at a third location connected to the network to receive one of raw sleep-data and scored sleep-data.

18. A system of claim 1, wherein the analyzer at one or more other locations further comprises:
an interpreter at a third location connected to the network to receive commands and issue an interpretation to authorized recipients on the network.

19. A system of claim 1, wherein the analyzer at one or more other locations further comprises:
a refinement analysis at a third location connected to the network to receive the scored sleep-data score, analyze the scored sleep-data score and issue a treatment refinement analysis to an authorized recipient on the network.

20. A system of claim 1, wherein the analyzer at one or more other locations further comprises:
a refinement analysis at a third location connected to the network to receive raw sleep-data, analyze the raw sleep-data, and issue a treatment refinement analysis to an authorized recipient on the network.

21. A system of claim 3, wherein the virtual sleep-lab connectivity device provides one of audio communication, video communication, text communication, and iconic communication between the patient and a healthcare provider over the network.

22. A sleep-lab data interface comprising:
a central processor;
a network; and
a database for storing and retrieval of sleep-lab data.

23. An interface of claim 22, wherein the central processor comprises:
a server; and
a user authentication interface to grant or deny access to one or more other interfaces.

24. An interface of claim 22, wherein the database maintains one or more of patient information, patient sleep-lab raw data, a patient's scored sleep-data, a patient's interpreted sleep-data, a patient's follow-up analysis, version data on sleep-lab equipment, device driver data on sleep-lab equipment, and other relevant patient sleep diagnosis and treatment data.

25. An interface of claim 23, wherein the server facilitates real-time communication between a biometric sleep monitoring device and a patient monitoring facility.

26. An interface of claim 23, wherein the server facilitates real-time communication between a patient monitoring facility and one of a patient diagnosis facility and a patient analysis facility.

27. An interface of claim 23, wherein the server facilitates real-time communication between a patient analysis facility and a patient diagnosis facility.

28. An interface of claim 23, wherein the server facilitates batch communication between a biometric sleep monitoring device and one of a patient monitoring facility, a patient diagnosis facility and a patient analysis facility.

29. An interface of claim 23, wherein the server facilitates batch communication between a patient monitoring facility and one of a patient diagnosis facility and a patient analysis facility.

30. An interface of claim 23, wherein the server facilitates batch communication between a patient analysis facility and a patient diagnosis facility.

31. An interface of claim 23, wherein the server facilitates communication in the form of alerts.

32. An interface of claim 23, wherein the server facilitates data conversion to a standard data format.

33. A method of performing sleep studies at a remote location through the Internet, comprising:
communicating patient sleep-data through the Internet to a centralized server; and
accessing the centralized server to evaluate the patient sleep-data.

34. The method of claim 33, further comprising alerting a patient as to abnormalities in the patient sleep-data.

35. The method of claim 33, further comprising monitoring a patient to determine the patient sleep-data.

36. The method of claim 33, wherein accessing comprises scoring the patient sleep-data based on one or more attributes of availability, file name, file size, length of recording, date of recording, time of recording, place of recording, type of recording, recording equipment used, patient, doctor, sleep-lab, completeness, reviewer, review status, sequence number and datamap position.

37. A sleep lab system, comprising:
a sleep monitor configured for monitoring a patient's sleep-data;
server connectivity to the sleep monitor; and
a plurality of access ports interfacing through the server connectivity to the sleep monitor to access the data from the sleep monitor.

38. The sleep lab system of claim 37, wherein the server connectivity is configurable to a plurality of sleep monitor types.

39. A sleep-analysis software product, comprising:
a computer readable medium comprising instructions for monitoring a patient's sleep and for analyzing substantially real-time data resulting from monitoring.

40. The sleep analysis software product of claim 39, the sleep analysis software product configurable with a personal computer, wherein the instructions are modifiable through software downloads via the internet.

41. A process for CPAP follow-up, comprising:
receiving a patient's CPAP follow-up attributes from the patient's locale;
storing the attributes in a centralized server via a VSL network hub in response to receiving; and
evaluating the attributes to determine one or more of
whether the patient is adhering to the CPAP follow-up and
whether monitoring is operationally functional.

42. The process of claim 41, the patient CPAP attributes including one or more of quality of life information, hours used, and relative patient experiences.

43. The process of claim 41, further comprising transferring a questionnaire from the server to the patient for ascertaining the patient attributes.

44. The process of claim 41, further comprising transferring a questionnaire completed by the patient from the patient's locale to the centralized server.

45. The process of claim 44, wherein transferring comprises transferring the questionnaire to the server through one or more of an internet connection, a dial-up connection, a wireless device, and a cable connection.

46. The process of claim 44, wherein transferring comprises transferring the questionnaire through one of an audio and a video connection.

47. The process of claim 41, further comprising retrieving evaluation scores at the patient's locale through an internet connection.

48. The process of claim 41, further comprising modifying software at the patient's locale via the centralized server, the software operable to receive the patient's CPAP follow-up attributes, to store the attributes in a centralized server, and to evaluate the attributes.
